(19) United States
(12) Patent Application Publication
(10) Pub. No.: US 2004/0019271 A1
(43) Pub. Date: Jan. 29, 2004

(54) Pole pieces for magnetic resonance imaging systems

(75) Inventors: Judson Sloan Marte, Wynantskill, NY (US); Mark Gilbert Benz, Lincoln, VT (US); Peter George Frishmann, Phoeniz, AZ (US); William Daniel Barber, Ballston Lake, NY (US); Howard Roscoe Hart JR., Schenectady, NY (US); Juliana Chiang Shei, Niskayuna, NY (US); Luana Emiliana Lorio, Clifton Park, NY (US); Bulent Aksel, Clifton Park, NY (US)

Correspondence Address: GENERAL ELECTRIC COMPANY, GLOBAL RESEARCH CENTER, PATENT DOCKET RM. 4A59, PO BOX 8, BLDG. K-1 ROSS, NISKAYUNA, NY, 12309, US

(73) Assignee: General Electric Company, Niskayuna, NY

(21) Appl. No.: 10/064,587

(22) Filed: Jul. 29, 2002

(51) Int. Cl. A61B 5/05

(52) U.S. Cl. 600/410

(57) ABSTRACT

Pole pieces for magnetic resonance imaging systems comprise a plurality of laminated sheets of an alloy comprising iron and aluminum. Such an alloy can comprise up to about 17 weight percent aluminum and can further comprise cobalt, nickel, and/or silicon. A sheet of iron-aluminum alloy for such pole pieces has a resistivity greater than about 60 mico-ohm.cm. A sheet is formed in a process comprising hot forging, hot rolling, and cold rolling. The process can further comprise annealing before and/or after the step of cold rolling.

### BACKGROUND OF INVENTION

The present invention relates to pole pieces for magnetic resonance imaging systems. In particular, the present invention relates to such pole pieces comprising laminates of magnetic materials comprising an iron alloy containing aluminum. The present invention also relates to methods for making such pole pieces.

Magnetic resonance imaging (“MRI”) is a technique for obtaining a tomographic image of an internal portion of a person or an object. It is necessary to form a stable and intense magnetic field in the magnetic field generating device of a MRI system in order to obtain a clear tomographic image.

One embodiment of a typical magnetic field generating device for a MRI system is schematically illustrated in FIG. 1, showing the major components thereof. Two permanent magnets 1 are disposed opposite to each other in a magnetic field generating source. One side of a permanent magnet 1 is secured to a yoke 3, and an opposed side is connected to a pole piece 2. Pole pieces 2 are also made of a magnetic material and have a function of shaping and enhancing the uniformity of the magnetic field in space 4 between pole pieces 2. Gradient magnetic field coils 6 disposed in the vicinity of each of magnetic pole pieces 2 usually comprise three sets of coil groups corresponding to three directions X, Y, and Z for obtaining positional information in space 4. When a time-varying current is applied to selected gradient magnetic field coils 6 a time-varying gradient magnetic field is generated in a desired direction. Magnetic pole pieces 2 typically have a shape of plates, and eddy current tends to be generated within them, leading to undesirable effects in the magnetic field. In order to reduce the eddy current, pole pieces have been made of laminations of thin sheets of soft magnetic materials, such as permalloy, amorphous iron, or Si—Fe alloy. Thinner sheets and higher resistivity are favored as they produce fewer eddy currents. However, there are limitations associated with prior-art materials used to make pole pieces. For example, although the resistivity of Si—Fe alloys can be increased with increasing content of silicon, they become brittle, and thus difficult to be formed into thin sheets, as their silicon content increases much higher than about 3.5 weight percent.

Therefore, there is a continued need to provide materials that can be formed into thinner sheets from materials having higher resistivity, thereby, having low eddy current loss for MRI pole pieces. In addition, it is very desirable to provide such pole pieces to improve the image quality of MRI systems.

### SUMMARY OF INVENTION

The present invention provides materials having desirable ductility and resistivity for magnetic pole pieces of MRI systems and MRI magnetic pole pieces comprising such materials.

In one aspect of the present invention, the material comprises an iron-aluminum alloy. It is to be understood that the term “iron-aluminum alloy” means an alloy the major components of which are iron and aluminum. Such an alloy can contain amounts of other components, which may desirably or may not substantially and adversely affect a magnetic or electrical property of the alloy of the major components. Typically, minor components that do not adversely affect a desirable property of the alloy of the major components may be present in an amount less than about 0.1 weight percent. For example, such an alloy can contain incidental amounts of other components that are unavoidable in the process of producing iron or aluminum. Thus, an alloy disclosed herein enumerates only its major components.

In another aspect of the present invention, the material comprises an iron-aluminum-cobalt alloy, iron-aluminum-nickel alloy, or iron-aluminum-cobalt-nickel alloy.

In still another aspect of the present invention, the material comprises iron, aluminum, and at least a third component that beneficially modifies at least a magnetic property or an electrical resistivity of the alloy.

In still another aspect of the present invention, a magnetic pole piece for a MRI system comprises a plurality of sheets of an alloy comprising iron and aluminum; iron, aluminum, and cobalt; or iron, aluminum, and nickel which sheets are laminated together.

In still another aspect of the present invention, a MRI system comprises at least a pole piece that comprises a plurality of laminated sheets of an iron-aluminum, an iron-aluminum-cobalt alloy, or an iron-aluminum-nickel alloy.

In still another aspect of the present invention, a process for making sheets of an alloy comprising iron and aluminum comprises hot rolling and cold rolling a piece of the alloy to a desired thickness. The process can further comprise at least an annealing before or after a rolling.

Other features and advantages of the present invention will be apparent from a perusal of the following detailed description of the invention and the accompanying drawings in which the same numerals refer to like elements.

### BRIEF DESCRIPTION OF DRAWINGS

FIG. 1 shows a schematic diagram of a prior-art magnetic field generating device of a MRI system.

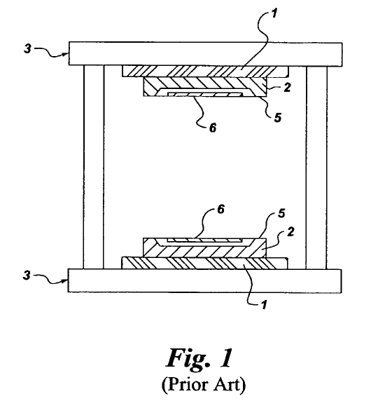

FIG. 2 shows magnetization curves for sheets of iron-aluminum alloy containing 6.5 weight percent aluminum.

FIG. 3 shows a magnetization curve at 40 Hz for a sheet of iron-aluminum alloy containing 6.5 weight percent aluminum and annealed at 1097° C. for one hour.

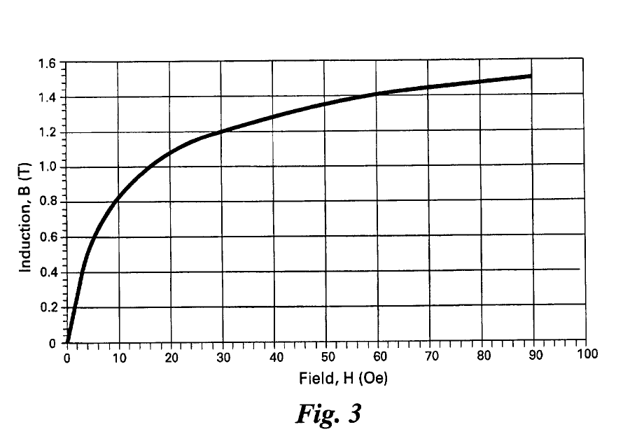

### DETAILED DESCRIPTION

One important desired property of a magnetic material is its low core loss. Core loss is the sum of the hysteresis loss and the eddy current loss, measured in W/kg (watts per kilogram). Low core loss is also desired for materials used to make pole pieces of a MRI system. Hysteresis loss is the irreversible energy loss incurred when the magnetization of a magnetic material is reversed. Hysteresis loss can be affected by selection of the composition of the material. Eddy current loss is the irreversible energy loss in the form of heat due to the formation of induced current in the magnetic material. Besides the undesired loss of energy, large eddy current also adversely affects the homogeneity of the magnetic field of a MRI system and retards the achievement of the maximum magnetic field and, thus, lowers the quality of the image of an object under examination. Eddy current can be reduced by using a material having higher resistivity that is formed into thin sheets. Lower core loss can further be achieved with lower coercivity and/or modification of the grain orientation; e.g., two magnetically “easy” crystal directions lie in the plane of the sheet.

Materials, such as amorphous iron and non-oriented silicon steel, have been used to make pole pieces for MRI systems. These materials have certain disadvantages. Amorphous iron is expensive. Steel containing a small amount of silicon may be made into thin sheets. However, in attempts to increase its resistivity by increasing the silicon content beyond about 3.5 weight percent, silicon steel becomes brittle and loses its ductility and, consequently, its ability to be formed into thin sheets. Processes developed to add Si to already thin Si—Fe sheets are expensive.

The present invention provides alloys comprising iron and aluminum and having desirable ductility and resistivity suitable for magnetic pole pieces of MRI systems and MRI magnetic pole pieces comprising such alloys. An iron-aluminum alloy for magnetic pole pieces of the present invention comprises from about 0.5 weight percent up to about 17 weight percent aluminum. Preferably, the alloy comprises an aluminum content less than or equal to about 10 weight percent. An alloy comprising an aluminum content less than or equal to 17 weight percent may be made from an alloy having a higher aluminum content by melting together appropriate amounts of substantially pure iron and such a higher aluminum-content iron-aluminum alloy. Further additions of other elements may be made to modify the microstructure of the alloy without detrimentally affecting the processability of the alloy to thin sheets. An alloy comprising iron and aluminum for making pole pieces of MRI systems may contain other incidental minor components that unavoidably accompany the process of making aluminum or iron or that are present in the raw materials of the alloy as long as they do not adversely affect the desired property of the final alloy. For example, these minor components may be phosphorus, sulfur, carbon, hydrogen, oxygen, nitrogen, rare-earth metals, or other metals, such as manganese, copper, chromium, or molybdenum. Typically, each of these minor components should be present in an amount less than about 0.1 weight percent, preferably less than bout 0.05 weight percent, more preferably less than about 0.01 weight percent, and most preferably less than about 0.005 weight percent.

In another aspect of the present invention, the iron-aluminum alloy can further comprise at least an element that beneficially modifies at least a magnetic property or an electrical property of the starting iron-aluminum alloy. For example, in one embodiment of the present invention, MRI pole pieces are made of an alloy comprising iron, aluminum, and cobalt. Addition of cobalt counteracts the loss of saturation magnetization of the alloy as the aluminum content increases. In another embodiment, nickel can be added to iron-aluminum alloy beneficially to affect the permeability of the starting iron-aluminum alloy. Such an iron-aluminum-cobalt alloy or iron-aluminum-nickel alloy can comprise from about 0.1 weight percent up to about 10 weight percent cobalt or nickel. Other minor components disclosed above may be present in the iron-aluminum-cobalt alloy as long as they do not adversely affect the desired property of the alloy.

In another embodiment of the present invention, the iron-aluminum alloy can comprise silicon in an amount from about 0.1 percent by weight to about 4 percent by weight. In still another embodiment, the alloy comprises iron, aluminum, silicon, and cobalt or nickel, in proportions disclosed above.

The present invention provides pole pieces of a MRI system. A pole piece of the present invention comprises a plurality of sheets of an iron-aluminum alloy, an iron-aluminum-cobalt alloy, or an iron-aluminum-nickel alloy, which sheets are laminated together into a stack using a binder such as a polymeric material. Each sheet has a thickness less than about 0.5 mm, preferably less than about 0.3 mm, and more preferably less than about 0.2 mm. Organic or inorganic binders may be used in producing laminated pole pieces. Suitable organic binders are epoxy resins and acrylic resins. Suitable inorganic binders are silicates or residues of organo metallic compounds resulting from a decomposition thereof. For example, polyorganosilanes or polyorganosiloxanes can leave residues comprising silicon carbide or silicon oxycarbide. Polysilazanes and silicon polymers with {—Si—N—} bonds can leave residues comprising silicon nitride or silicon carbonitride. Such binders or residues thereof are preferably electrically insulating.

Sheets of non-oriented or oriented iron alloy containing aluminum can be used in the present invention to produce pole pieces for MRI systems. In one aspect of the present invention, a sheet can comprise a doubly-oriented alloy in which the grains are oriented with a cube plane of the unit cell parallel to the surface of the sheet.

A sheet having a thickness less than about 0.5 mm is made by a process comprising one or more steps of hot rolling and cold rolling a slab or an ingot of alloy to a desired thickness. Each of the step of hot rolling or cold rolling can comprise a plurality of passes through the mill to reduce the thickness in increments in the range from about 10 to about 20 percent. The process can further comprise at least an annealing before or after a hot or cold rolling. Typically, an annealing is carried out in a reducing atmosphere, such as hydrogen, or an inert atmosphere such as argon, neon, helium, krypton, xenon, or a mixture thereof. Annealing may also be carried out in a vacuum. After an annealing, the alloy article is typically cooled down slowly in the same atmosphere substantially to room temperature.

In one embodiment of the process, an iron-aluminum alloy was prepared by melting in an electric furnace under a vacuum, a reducing atmosphere such as hydrogen, or an inert atmosphere, such as helium, neon, argon, krypton, xenon, or a mixture thereof, desired proportions of substantially pure iron and aluminum. Iron may be melted first, and aluminum is added thereto. Alternatively, an iron-aluminum alloy having a high content of aluminum may be melted, such as greater than about 17 weight percent aluminum, and substantially pure iron is added thereto to achieve the desired composition.

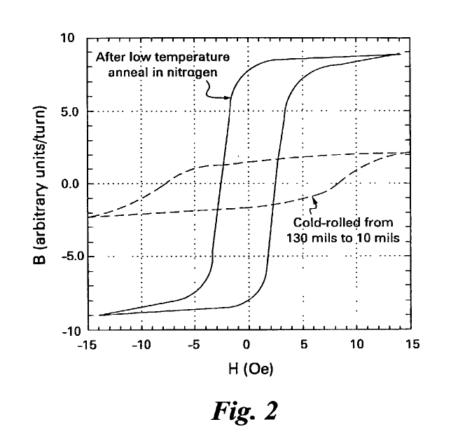

The present inventors discovered unexpectedly that while iron alloys containing 4 weight percent or higher silicon were very brittle, iron-aluminum alloys are extremely ductile, even at liquid nitrogen temperature. For example, one iron alloy containing about 6 weight percent silicon and another containing about 4 weight percent silicon and 6 weight percent aluminum were readily crushed into coarse powder using a jaw crusher and a disc mill. On the contrary, an iron alloy containing 6.5 weight percent aluminum was not crushable at room temperature. Another piece of the same iron-aluminum alloy suffered only a dent after it was cooled to liquid nitrogen temperature for one hour and hit with a ball-peen hammer. Therefore, very thin sheets of magnetic materials could be produced from iron-aluminum alloys. Other components can also be added to these alloys to desirably modify their basic properties for specific applications. Sheets of iron aluminum alloy containing 6.5 weight percent aluminum were produced having a length of about 91 cm and a thickness of about 0.25 mm and were coated with MgO. The resistivity of the sheet was 70 microohm-cm. Coils were made with sheets that were unannealed or annealed in nitrogen at about 760° C. for four hours and cooled to about 370° C. at a rate of about 55° C./hour. The sheets were formed into coils having a diameter of about 7.6 cm. FIG. 2 shows magnetization curves for the coils.

Sheets having a width of about 5 cm and a thickness of about 0.2 mm of another sample of iron-aluminum alloy containing 6.5 weight percent aluminum were made and subject to various annealing temperatures under a reducing atmosphere containing hydrogen. The annealing consisted of raising the temperature rapidly to about 750° C., ramping to the annealing temperature at about 50° C./hour, holding at the annealing temperature for an annealing time, cooling to about 750° C. at about 50° C./hour, then further cooling the furnace to near room temperature at an uncontrolled rate. The coercivity of the annealed sheets was measured and is shown in Table 1. It is possible that the annealing time can be extended beyond 8 hours, such as up to about 24 hours or longer, to realize further reduction in coercivity.

TABLE 1

| Annealing | | | |
|---|---|---|---|
| Temperature | Annealing Time | DC Coercivity | 1 kHz Coercivity |
| (° C.) | (hour) | (Oe) | (Oe) |
| 815 | 1 | 0.98 | 1.61 |
| 815 | 8 | 0.87 | 1.60 |
| 995 | 4.5 | 0.47 | 1.45 |
| 995 | 4.5 | 0.44 | 1.58 |
| 995 | 4.5 | 0.52 | 1.57 |
| 1045 | 8 | 0.51 | 1.53 |
| 1175 | 1 | 0.55 | 1.03 |
| 1175 | 8 | 0.57 | 1.00 |
| 1175 | 1 | 0.55 | 1.36 |
| 1175 | 8 | 0.57 | 1.32 |

### EXAMPLE

A cast ingot of iron-aluminum alloy having the desired composition (about 4, 6, 8, and 10 weight percent aluminum) was cut into pieces about 23 cm tall and about 9 cm on each side and was hot worked to break up the cast grain structure. The hot working consisted of forging and rolling at temperatures between 1300° C. and 900° C., the temperature gradually being decreased to create a refined grain structure. The final thickness after hot rolling was 3 mm. The material was then cold rolled to less than 0.5 mm using reductions of about 10-20% per pass. It is envisioned that reductions of up to about 50% per pass may be possible. The alloys containing about 8 and about 10 weight percent aluminum were annealed at about 900° C. for one hour before cold rolling. Longer annealing times, such as 24 hours or longer, may be desirable in specific instances. Another annealing temperature in the range from about 900° C. to about 1050° C. may also be used. This annealing resulted in cold rolling to a thickness of about 0.8 mm or about 0.4 mm using 10% reductions without difficulty. The sheets were machined into tensile specimens consistent with test method ASTM E8-99 for tensile strength measurement, rings (about 5-cm outer diameter and about 4-cm inner diameter) for core loss measurement, and Epstein strips (about 3 cm wide and about 30 cm long) for magnetic loss measurement. All of the specimens, rings, and Epstein strips were coated by hand with a coating of MgO and heat-treated before testing. The MgO coating used is similar to the C-2 inorganic mill coating used commercially. The rings underwent various heat treatment at temperatures in the range from about 900° C. to about 1050° C. for 1-5 hours. Additional rings of iron-aluminum alloy having 4 and 8 weight percent aluminum were also heat-treated at about 1200° C. for 5 hours. The tensile specimens and Epstein strips were heat-treated at about 975° C. for 3 hours before testing. Samples of silicon steel (having about 3.6 weight percent silicon) were also processed and tested for comparison.

A step of annealing may be carried out at a substantially constant temperature or the temperature may be ramped up from ambient to the final temperature, and the specimen is held at the final temperature for a desired duration.

Testing Procedures:

Stacks of laminated rings wound with an insulating tape and wire coils having a height of about 8 mm were tested for core loss and permeability using Model SMT-600 magnetic tester (KJS Associates Inc., Indianapolis, Ind.) using ASTM standard A912-3 (1998).

Tensile strength and Rockwell B hardness (“HRB”) were measured according to ASTM standards E8-99 (1998) and E18-98 (1999), respectively. The tensile testing was performed at room temperature and ambient pressure with a speed of about 5 mm/minute.

Prior to the application of the MgO coating, as-machined Epstein strips were tested for resistivity using a Keithley 580 four-probe micro-ohmmeter according to ASTM standard A712-97 (1997).

The results of testing for mechanical properties and resistivity are shown in Table 2.

TABLE 2

| | | | Ultimate | |
|---|---|---|---|---|
| | | Young's | Tensile | |
| | | Modulus | Strength | Resistivity |
| Alloy | HRB | (10³ MPa) | (MPa) | (μΩ.cm) |
| Fe-Si | 78 | 1371 | 439 | 52 |
| (3.6 wt. % Si) | | | | |
| Fe-Al | 55 | — | — | 61 |
| (4 wt. % Al) | | | | |
| Fe-Al | 76 | 1266 | 376 | 75 |
| (6 wt. % Al) | | | | |
| Fe-Al | 80 | — | — | 92 |
| (8 wt. % Al) | | | | |
| Fe-Al | 82 | 1641 | 463 | 104 |
| (10 wt. % Al) | | | | |

The results of testing for magnetic properties are shown in Table 3.

TABLE 3

| | | | | | Core | Calculated | Induc- |
|---|---|---|---|---|---|---|---|
| | | | | | Loss | Eddy | tion |
| | | | | | (W/kg | Current | (at 60 |
| | Sheet | | Heat Treatment | | at 60 | Loss | Hz, |
| | Thick- | Spec- | Tem- | | Hz | (W/kg at | 50 |
| | ness | imen | perature | Time | and | 60 Hz and | Oe) |
| Alloy | (mm) | Type | (° C.) | (hr) | 1 T) | 1 T) | (T) |
| Fe-Si | 0.8 | Ring | 1175 | 3 | 2.56 | — | 1.57 |
| 3.6 | | | | | | | |
| wt. % | | | | | | | |
| Si) | | | | | | | |
| Fe-Al | 0.4 | Ring | 1050 | 5 | 1.89 | 0.63 | 1.51 |
| (4 wt. | 0.4 | Ring | 1200 | 5 | 1.61 | 0.35 | 1.50 |
| % Al) | | | | | | | |
| Fe-Al | 0.8 | Ring | 900 | 1 | 3.37 | 0.89 | 1.43 |
| (6 wt. | 0.8 | Ring | 900 | 5 | 3.23 | 0.78 | 1.44 |
| % Al) | 0.8 | Ring | 1050 | 1 | 2.86 | 0.78 | 1.44 |
| | 0.8 | Ring | 1050 | 5 | 3.13 | 1.11 | 1.42 |
| Fe-Al | 0.4 | Ring | 900 | 1 | 2.00 | 0.32 | 1.44 |
| (6 wt. | 0.4 | Ring | 900 | 5 | 2.16 | 0.39 1.39 | |
| % Al) | 0.4 | Ring | 1050 | 1 | 2.03 | 0.37 | 1.40 |
| | 0.4 | Ring | 1050 | 5 | 1.86 | 0.44 | 1.42 |
| Fe-Al | 0.4 | Ring | 1050 | 5 | 2.42 | 0.54 | 1.39 |
| (8 wt. | 0.4 | Ring | 1200 | 5 | 2.25 | 0.61 | 1.37 |
| % Al) | | | | | | | |
| Fe-Al | 0.4 | Ring | 900 | 1 | — | — | — |
| (10 | 0.4 | Ring | 900 | 5 | 2.49 | 0.35 | 1.37 |
| wt. % | | | | | | | |
| Al) | 0.4 | Ring | 1050 | 1 | 2.41 | 0.43 | 1.37 |
| | 0.4 | Ring | 1050 | 5 | 2.28 | 0.41 | 1.39 |

Sheets of an alloy comprising iron and aluminum, each having a thickness of less than about 0.5 mm, preferably less than about 0.3 mm, more preferably less than about 0.2 mm, and most preferably less than about 0.1 mm, are laminated together into a stack using a binder to form improved pole pieces for MRI systems. The lamination can be carried out at room temperature or at a temperature that does not prevent a handling of the piece. If a solid organic binder is used, a temperature such as its melting temperature may be used for lamination. A lamination temperature may also be one that promotes a polymerization of an organic resin binder. Furthermore, the stack may be heat-treated at a temperature that decomposes an organic or organo-metallic binder to leave a temperature-resistant ceramic or inorganic residue. After lamination, the stack may be cut or otherwise shaped into a desired shape and dimensions for incorporating into a MRI system. Lamination may be carried out under an applied pressure, such as up to 100 MPa, if desired. After lamination, the stack may be advantageously annealed at a temperature in the range from about 900° C. to about 1300° C., preferably from about 1000° C. to about 1200° C. to relieve stress. Construction of pole pieces such as one of the methods disclosed in U.S. Pat. Nos. 5,283,544; 6,150,818; and 6,150,819, the contents of which are incorporated herein by reference, may be used with an alloy disclosed herein to provide improved pole pieces for MRI systems. One embodiment of a pole piece of the present invention comprises a plurality of stacks of laminated sheets. The stacks are attached to each other to complete a thickness of the pole piece such that the rolling direction of the sheets in one stack are oriented at an angle relative to the sheets in an adjacent stack. In one embodiment, the angle is 90 degrees.

While various embodiments are described herein, it will be appreciated from the specification that various combinations of elements, variations, equivalents, or improvements therein may be made by those skilled in the art, and are still within the scope of the invention as defined in the appended claims.

1. A magnetic pole piece for a magnetic resonance imaging (“MRI”) system, said pole piece comprising a stack of a plurality of sheets of an alloy that comprises iron and aluminum, said sheets being laminated together.

2. The magnetic pole piece for a MRI system according to claim 1, wherein said alloy comprises from about 0.5 to about 17 weight percent aluminum.

3. The magnetic pole piece for a MRI system according to claim 2, wherein said alloy further comprises cobalt in an amount from about 0.1 weight percent to about 10 weight percent.

4. The magnetic pole piece for a MRI system according to claim 2, wherein said alloy further comprises nickel in an amount from about 0.1 weight percent to about 10 weight percent.

5. The magnetic pole piece for a MRI system according to claim 2, wherein said alloy further comprises silicon in an amount from about 0.1 weight percent to about 4 weight percent.

6. The magnetic pole piece for a MRI system according to claim 3, wherein said alloy further comprises silicon in an amount from about 0.1 weight percent to about 4 weight percent.

7. The magnetic pole piece for a MRI system according to claim 4, wherein said alloy further comprises silicon in an amount from about 0.1 weight percent to about 4 weight percent.

8. The magnetic pole piece for a MRI system according to claim 1, wherein each of said sheets has a thickness less than about 0.5 mm.

9. The magnetic pole piece for a MRI system according to claim 1, wherein each of said sheets has a thickness less than about 0.3 mm.

10. The magnetic pole piece for a MRI system according to claim 1 , wherein each of said sheets has a thickness less than about 0.1 mm.

11. The magnetic pole piece for a MRI system according to claim 1, wherein said sheets are separated from each other by an electrically insulating material.

12. The magnetic pole piece for a MRI system according to claim 10, wherein said electrically insulating material is selected from the group consisting of inorganic and organic electrically insulating materials.

13. The magnetic pole piece for a MRI system according to claim 10, wherein said electrically insulating material is a silicate.

14. The magnetic pole piece for a MRI system according to claim 10, wherein said electrically insulating material is an organic polymeric material selected from the group consisting of epoxy resins, acrylic resins, polyorganosilanes, polyorganosiloxanes, polysilazanes, silicon polymers that have —Si—N— bonds, and mixtures thereof.

15. The magnetic pole piece for a MRI system according to claim 10, wherein said electrically insulating material is a residue resulting from a heat treatment of a polymer selected from the group consisting of polyorganosilanes, polyorganosiloxanes, polysilazanes, silicon polymers that have —Si—N— bonds, and mixtures thereof.

16. The magnetic pole piece for a MRI system according to claim 1, wherein said sheets have a resistivity greater than about 60 micro-ohm.cm, as measured according to ASTM standard A712-97.

17. The magnetic pole piece for a MRI system according to claim 1, wherein said sheets are laminated at a pressure up to about 100 MPa.

18. The magnetic pole piece for a MRI system according to claim 1, wherein said sheets are laminated with an organic binder at a melting temperature thereof.

19. The magnetic pole piece for a MRI system according to claim 18, wherein said sheets have been annealed before being laminated together.

20. The magnetic pole piece for a MRI system according to claim 1, wherein said stack is annealed at a temperature from about 900° C. to about 1300° C.

21. The magnetic pole piece for a MRI system according to claim 1, wherein said stack is annealed at a temperature from about 1000° C. to about 1200° C. in an environment selected from the group consisting of vacuum, reducing gas atmosphere, inert gas atmosphere, and combinations thereof.

22. A magnetic pole piece for a MRI system, said pole piece comprising a plurality of stacks, each of said stack comprising a plurality of sheets of an alloy that comprises iron and aluminum, said sheets being laminated together, said stacks being disposed adjacent one another such that sheets of one stack are oriented at an angle relative to sheets of an adjacent stack.

23. A magnetic pole piece for a MRI system, said pole piece comprising a stack of a plurality of sheets of an alloy that comprises iron and aluminum, said sheets being laminated together, each of said sheet being formed in a process comprising:
forging a block of said alloy at a temperature in a range from about 1000° C. to about 1300° C. to produce a forged alloy;
hot rolling said forged alloy at a temperature of about 1300° C. to produce a hot-rolled body; and
cold rolling said hot-rolled body using a thickness reduction increment from about 10 percent to about 50 percent to produce said sheet.

24. The magnetic pole piece for a MRI system according to claim 23 further comprising performing a first annealing of said hot-rolled body at a temperature from about 900° C. to about 1050° C. before said cold rolling.

25. The magnetic pole piece for a MRI system according to claim 24, wherein said annealing is carried out for a time between about one hour and about 24 hours.

26. The magnetic pole piece for a MRI system according to claim 23 further comprising performing a second annealing of said sheet after said cold rolling, said annealing being carried out in an environment selected from the group consisting of vacuum, reducing gas, inert gas, and combinations thereof.

27. The magnetic pole piece for a MRI system according to claim 26 further comprising performing a second cold rolling after said second annealing.

28. A MRI system comprising at least a magnetic pole piece that comprises a stack of a plurality of sheets of an alloy that comprises iron and aluminum, said sheets being laminated together.

29. The MRI system according to claim 28, wherein said alloy comprises from about 0.5 to about 17 weight percent aluminum.

30. The MRI system according to claim 28, wherein said alloy comprises from about 0.5 to about 10 weight percent aluminum.

31. The MRI system according to claim 28, wherein each of said sheets has a thickness less than about 0.5 mm.

32. The MRI system according to claim 29, wherein said alloy further comprises cobalt in an amount from about 0.1 weight percent to about 10 weight percent.

33. The MRI system according to claim 29, wherein said alloy further comprises nickel in an amount from about 0.1 weight percent to about 10 weight percent.

34. The MRI system according to claim 29, wherein said alloy further comprises silicon in an amount from about 0.1 weight percent to about 4 weight percent.

35. The MRI system according to claim 32, wherein said alloy further comprises silicon in an amount from about 0.1 weight percent to about 4 weight percent.

36. The MRI system according to claim 33, wherein said alloy further comprises silicon in an amount from about 0.1 weight percent to about 4 weight percent.

37. The MRI system according to claim 28, wherein each of said sheets has a thickness less than about 0.5 mm.

38. The MRI system according to claim 28, wherein each of said sheets has a thickness less than about 0.3 mm.

39. The MRI system according to claim 28, wherein each of said sheets has a thickness less than about 0.1 mm.

40. The MRI system according to claim 28, wherein said sheets are separated from each other by an electrically insulating material.

41. The MRI system according to claim 40, wherein said electrically insulating material is selected from the group consisting of inorganic and organic electrically insulating materials.

42. The MRI system according to claim 40, wherein said electrically insulating material is a silicate.

43. The MRI system according to claim 40, wherein said electrically insulating material is an organic polymeric material selected from the group consisting of epoxy resins, acrylic resins, polyorganosilanes, polyorganosiloxanes, polysilazanes, silicon polymers that have —Si—N— bonds, and mixtures thereof.

44. The MRI system according to claim 40, wherein said electrically insulating material is a residue resulting from a heat treatment of a polymer selected from the group consisting of polyorganosilanes, polyorganosiloxanes, polysilazanes, silicon polymers that have —Si—N— bonds, and mixtures thereof.

45. The MRI system according to claim 28, wherein said sheets have a resistivity greater than about 60 micro-ohm.cm, as measured according to ASTM standard A712-97.

46. The MRI system according to claim 28, wherein said sheets are laminated at a pressure up to about 100 MPa.

47. The MRI system according to claim 28, wherein said sheets are laminated with an organic binder at a melting temperature of said organic binder.

48. The MRI system according to claim 28, wherein said stack is annealed at a temperature from about 900° C. to about 1300° C.

49. The MRI system according to claim 28, wherein said stack is annealed at a temperature from about 1000° C. to about 1200° C.

50. The MRI system according to claim 28, wherein said stack is annealed in an environment selected from the group consisting of vacuum, reducing gas, inert gas, and combinations thereof.

51. A process for making sheets of a magnetic alloy comprising iron and aluminum, said process comprising:
forging a block of said alloy at a temperature in a range from about 1000° C. to about 1300° C. to produce a forged alloy;
hot rolling said forged alloy at a temperature of about 1300° C. to produce a hot-rolled body; and
cold rolling said hot-rolled body using a thickness reduction increment from about 10 percent to about 50 percent to produce said sheet.

52. The process for making sheets of a magnetic alloy comprising iron and aluminum according to claim 51 further comprising performing a first annealing of said hot-rolled body at a temperature from about 900° C. to about 1050° C. before said cold rolling.

53. The process for making sheets of a magnetic alloy comprising iron and aluminum according to claim 52, wherein said annealing is carried out for about one hour.

54. The process for making sheets of a magnetic alloy comprising iron and aluminum according to claim 52 further comprising performing a second annealing of said sheet after said cold rolling.

55. The process for making sheets of a magnetic alloy comprising iron and aluminum according to claim 54 further comprising performing a second cold rolling after said second annealing.
